(19) United States
(12) Patent Application Publication
(10) Pub. No.: US 2026/0207931 A1
(43) Pub. Date: Jul. 23, 2026

(54) COMPOSITIONS AND METHODS FOR USING ALTERNATING ELECTRIC FIELDS AND FOLFIRINOX

(71) Applicant: NOVOCURE GMBH, Baar (CH)

(72) Inventors: Eyal Dor-On, Haifa (IL); Hila Ene, Haifa (IL)

(21) Appl. No.: 19/455,048

(22) Filed: Jan. 21, 2026

Related U.S. Application Data
(60) Provisional application No. 63/747,717, filed on Jan. 21, 2025.

(51) Int. Cl. A61N 1/36; A61K 31/4375; A61K 31/513; A61K 31/522; A61K 31/555; A61P 35/04

(52) U.S. Cl. CPC A61N 1/36002; A61K 31/4375; A61K 31/513; A61K 31/522; A61K 31/555; A61P 35/04

(57) ABSTRACT

Disclosed are method of treating a subject in need thereof comprising applying an alternating electric field, at a frequency for a period of time, to a target site of the subject in need thereof; and administering a combination of leucovorin calcium, fluorouracil, irinotecane hydrochloride, and oxaliplatin to the subject in need thereof. Disclosed are methods of reducing viability of cancer cells comprising applying an alternating electric field, at a frequency for a period of time, to a population of cancer cells; and contacting the population of cells with a combination of leucovorin calcium, fluorouracil, irinotecane hydrochloride, and oxaliplatin. Disclosed are methods of increasing apoptosis of cancer cells comprising applying an alternating electric field, at a frequency for a period of time, to a population of cancer cells; and contacting the population of cancer cells with a combination of leucovorin calcium, fluorouracil, irinotecane hydrochloride, and oxaliplatin. Disclosed are methods of reducing the number of cancer cells comprising applying an alternating electric field, at a frequency for a period of time, to a population of cancer cells; and contacting the population of cancer cells with a combination of leucovorin calcium, fluorouracil, irinotecane hydrochloride, and oxaliplatin.

### CROSS-REFERENCE TO RELATED APPLICATIONS

This application claims the benefit of U.S. Provisional Patent Application No. 63/747,717, filed on Jan. 21, 2025, which is incorporated by reference herein in its entirety.

### BACKGROUND

Despite improvements in cancer treatments, there remains a critical need to further improve therapies so as to prolong patients'lives while maintaining quality of life, particularly in the case of advanced cancers such as pancreatic cancers that often are, or become, resistant to current therapeutic modalities.

Incidence of pancreatic cancer has markedly increased during the past several decades. It now ranks as the fourth leading cause of cancer death in the United States. Pancreatic cancer's high mortality rate is due to a dearth of effective therapies and a complete absence of reliably durable therapies. Because of the location of the pancreas, pancreatic cancer is typically not diagnosed until a tumor has become large enough to produce systemic symptoms. This, coupled with the absence of good screening tools and a limited understanding of risk factors, results in patients usually having advanced disease, often advanced metastatic disease, at the time of diagnosis. Metastatic pancreatic cancer has a dismal prognosis and is almost uniformly fatal, with an overall survival rate of less than 4% at 5 years.

There are few approved treatment options for advanced or metastatic pancreatic cancers. Single-agent gemcitabine is the current standard of care in first-line treatment of advanced and metastatic pancreatic adenocarcinoma. In clinical trials, single-agent gemcitabine has consistently demonstrated a median prolongation of survival of 5 to 6 months and a 1-year survival rate of about 20%. Single agent gemcitabine was also approved as second line treatment for patients previously treated with but no longer responsive to 5-fluorouracil, with a median overall prolongation of survival of 3.9 months.

During the last few years, one combination chemotherapy regimen that has emerged as standard of care for first-line treatment of metastatic pancreatic cancer is the combination therapy of 5-fluorouricil (5-FU)/leucovorin (LV)+irinotecan+oxaliplatin (FOLFIRINOX). However, even with this treatment there is a need for improvements in, and effective alternatives to current therapies. The disclosed invention addresses this need by combining therapeutics with alternating electric fields.

### BRIEF SUMMARY

Disclosed are method of treating a subject in need thereof comprising applying an alternating electric field, at a frequency for a period of time, to a target site of the subject in need thereof; and administering a combination of leucovorin calcium, fluorouracil, irinotecane hydrochloride, and oxaliplatin to the subject in need thereof.

Disclosed are methods of reducing viability of cancer cells comprising applying an alternating electric field, at a frequency for a period of time, to a population of cancer cells; and contacting the population of cells with a combination of leucovorin calcium, fluorouracil, irinotecane hydrochloride, and oxaliplatin.

Disclosed are methods of increasing apoptosis of cancer cells comprising applying an alternating electric field, at a frequency for a period of time, to a population of cancer cells; and contacting the population of cancer cells with a combination of leucovorin calcium, fluorouracil, irinotecane hydrochloride, and oxaliplatin.

Disclosed are methods of reducing the number of cancer cells comprising applying an alternating electric field, at a frequency for a period of time, to a population of cancer cells; and contacting the population of cancer cells with a combination of leucovorin calcium, fluorouracil, irinotecane hydrochloride, and oxaliplatin.

Disclosed are methods of sensitizing BRCA wild-type cancer cells to treatment with leucovorin calcium, fluorouracil, irinotecane hydrochloride, and oxaliplatin comprising applying an alternating electric field, at a frequency for a period of time, to a population of cancer cells; and contacting the population of cancer cells with a combination of leucovorin calcium, fluorouracil, irinotecane hydrochloride, and oxaliplatin.

Disclosed are dosing regimens comprising applying an alternating electric field to a target site of a subject in need thereof; and administering a combination of leucovorin calcium, fluorouracil, irinotecane hydrochloride, and oxaliplatin to the subject in need thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the effects of one or more of TTFields (72 hrs, 1 V/cm, 150 kHz), leucovorin calcium, fluorouracil (5-FU), irinotecane hydrochloride (CPT-11), and oxaliplatin on the pancreatic cell line AsPC1. Top graph show results of cell count for each treatment and the additive effect is calculated by multiplying the cytotoxicity of TTFields at the intensity chosen per cell line with the cytotoxicity of chemotherapy alone. Middle graph are overall effect results calculated by multiplying the cytotoxicity observed by cell count with the colonogenic effect as calculated by colony assay results. Bottom graph are apoptosis results were evaluated in flow cytometry measured by Annexin V/PI staining. Also presented are titration graphs for each drug in AsPc cell line. Control is drugs only and the additive effect is measured by multiplying the cytotoxicity of TTFields at the intensity chosen per cell line with the cytotoxicity of chemotherapy alone.

FIG. 2 shows the effects of one or more of TTFields (72 hrs, 0.76 V/cm, 150 kHz), leucovorin calcium, fluorouracil, irinotecane hydrochloride, and oxaliplatin on the pancreatic cell line BxPC3. Top graph show results of cell count for each treatment and the additive effect is calculated by multiplying the cytotoxicity of TTFields at the intensity chosen per cell line with the cytotoxicity of chemotherapy alone. Middle graph are overall effect results calculated by multiplying the cytotoxicity observed by cell count with the colonogenic effect as calculated by colony assay results. Bottom graph are apoptosis results were evaluated in flow cytometry measured by Annexin V/PI staining. Also presented are titration graphs for each drug in BxPc3 cell line.

FIG. 3 shows the study design for the use of FOLFIRINOX and TTFields.

FIGS. 4A-4B show titration curves of 5-Fu (top), Leucovorin (middle) and oxaliplatin (bottom) in PDAC cell lines. FIG. 4A shows results from AsPC1 cells. FIG. 4B shows results from BxPC3 cells. From these titration curved the IC50 was established for further experiments in PDAC cell lines.

FIGS. 5A-5B show TTFields enhance the efficacy of FOLFIRINOX in PDAC cell lines. Top graphs represents cell count which was established by flow cytometry (FACS) to indicate cytotoxicity of treatment, middle graph represents an overall effects takes into account the clonogenic assay results multiplied by cytotoxicity to provide an overall treatment effect, and bottom graph shows results of apoptosis assays provided by Annexin V and 7AAD FACS analysis FIG. 5A shows results from AsPC1 cells. FIG. 5B shows results from BxPC3 cells.

FIGS. 6A-6B show TTFields modify the DNA damage repair pathways. FIG. 6A shows TTFields modify the expression of DNA damage repair pathways in a AsPC1 cell line. TTFields were applied for 24 and 48 hours, 1 V/cm, 150 kHz. FIG. 6B shows TTFields modify the expression of RNA and protein of DNA damage repair pathways in a AsPC1 cell line. TTFields were applied for 72 hours, 1 V/cm, 150 kHz. *P<0.05, **P<0.002, ***P<0.0002, ***P<0.0001, values represents RNA expression of DNA repair pathways difference from control.

FIGS. 7A-7B show TTFields modify the DNA damage repair pathways. FIG. 7A shows TTFields modify the expression of DNA damage repair pathways in a BxPC3 cell line. TTFields were applied for 24 and 48 hours, 1 V/cm, 150 kHz. FIG. 7B shows TTFields modify the expression of RNA and protein of DNA damage repair pathways in a BxPC3 cell line. TTFields were applied for 72 hours, 1 V/cm, 150 kHz. *P<0.05, **P<0.002, ***P<0.0002, ***P<0.0001, values represents RNA expression of DNA repair pathways difference from control.

FIG. 8 shows TTFields increase DNA damage induced by FOLFIRINOX in a AsPC1 cell line. Images were taken in confocal microscope for gamma-H2X staining (green) and nucleus (DAPI, blue) and results below were represented in the graph below analyzed with image J. The dosing was OxPl 0.36 uM, Irinotacan 0.39 uM, 5-FU 0.08 uM, LCVRN 5 uM. TTFields were applied 72 hrs, 1 V/cm, 150 kHz. One-way ANOVA, Sidak post-hoc, *P<0.05, **P<0.0002.

FIG. 9 shows TTFields increase DNA damage induced by FOLFIRINOX in a BxPC3 cell line. Images were taken in confocal microscope for gamma-H2X staining (green) and nucleus (DAPI, blue) and results below were represented in the graph below analyzed with image J The dosing was OxPl 0.4 uM, Irinotacan 0.51 uM, 5-FU 0.012 uM, LCVRN 5 uM. TTFields were applied 72 hrs, 0.76 V/cm, 150 kHz. One-way ANOVA, Sidak post-hoc, *P<0.05, **P<0.002.

FIG. 10 shows FOLFIRINOX induces S-phase arrest, while TTFields have mild effect on cell cycle in BxPC3 cell line. TTFields were applied at 1 V/cm, 150 kHz to AsPC1 or 0.76 V/cm, 150 kHz to BxPC3. For AsPC1 the dosing was OxPl 0.36 uM, Irinotacan 0.39 uM, 5-FU 0.08 uM, LCVRN 5 uM. For BxPC3 the dosing was OxPl 0.4 uM, Irinotacan 0.51 uM, 5-FU 0.012 uM, LCVRN 5 uM; *P<0.05; **P<0.002; ***P<0.0002; ****P<0.0001

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a subject” includes a plurality of such subjects, reference to “the cell” is a reference to one or more cells and equivalents thereof known to those skilled in the art, and so forth.

The word “or” as used herein means any one member of a particular list and also includes any combination of members of that list.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the pancreas. In some aspects, a cell or population of cells that can be a target site or a target cell include, but are not limited to, a cancer cell (e.g., a pancreatic cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g., a target site such as a cell). In some aspects, the alternating electrical field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with ovarian cancer or lung cancer.

By “treat” is meant to administer or apply a therapeutic, such as alternating electric fields and the combination of Leucovorin calcium, Fluorouracil, Irinotecane hydrochloride, Oxaliplatin, to a subject, such as a human or other mammal (for example, an animal model), that has cancer or has an increased susceptibility for developing cancer, in order to prevent or delay a worsening of the effects of cancer, or to partially or fully reverse the effects of cancer. For example, treating a subject having pancreatic cancer can comprise delivering a therapeutic to a cell in the subject.

By “prevent” is meant to minimize or decrease the chance that a subject develops cancer.

As used herein, the terms “administering” and “administration” refer to any method of providing one or more of Leucovorin calcium, Fluorouracil, Irinotecane hydrochloride, Oxaliplatin to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises contacting, exposing or applying. Thus, in some aspects, exposing a target site or subject to alternating electrical fields or applying alternating electrical fields to a target site or subject or contacting alternating electrical fields to a target site or subject means administering alternating electrical fields to the target site or subject. In some aspects, contacting, exposing and applying can be used interchangeably.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. Therapeutics

Disclosed are therapeutics, such as cancer therapeutics, that can be administered in combination with alternating electric fields.

Disclosed is a combination of therapeutics that includes leucovorin calcium, fluorouracil, irinotecane hydrochloride, and oxaliplatin.

In some aspects, Leucovorin calcium, or a derivative thereof, is administered to a subject or a population of cells. In some aspects, leucovorin calcium is an active metabolite of folic acid (also called folinic acid and citrovorum factor), which does not require metabolism by dihydrofolate reductase, the molecular target of folate antagonist-type chemotherapeutic drugs. Leucovorin calcium can counteract the toxic effects of these medications, ‘rescuing’ the patient while permitting the antitumor activity of the folate antagonist. In some aspects, Leucovorin calcium can also potentiate the effects of fluorouracil and its derivatives by stabilizing the binding of the drug's metabolite to its target enzyme, thus prolonging drug activity. In some aspects, the alternatives to leucovorin calcium can be leucovorin (Folinic acid) Calcium Pentahydrate, calcium folinate, or leucovorin. Thus, any of these can be used interchangeably with leucovorin calcium. In some aspects, the dose of leucovorin calcium can be 100-500 mg/m². In some aspects, the dose of leucovorin calcium 350 mg/m². In some aspects, the dose of leucovorin calcium 400 mg/m². In some aspects, any known therapeutic dose of leucovorin calcium can be used.

In some aspects, fluorouracil (5-FU), or a derivative thereof, is administered to a subject or a population of cells. In some aspects, fluorouracil is an antimetabolite fluoropyrimidine analog of the nucleoside pyrimidine with antineoplastic activity. Fluorouracil and its metabolites can possess a number of different mechanisms of action. In vivo, fluoruracil can be converted to the active metabolite 5-fluoroxyuridine monophosphate (F-UMP); replacing uracil, F-UMP incorporates into RNA and inhibits RNA processing, thereby inhibiting cell growth. In some aspects, another active metabolite, 5-5-fluoro-2′-deoxyuridine-5′-O-monophosphate (F-dUMP), inhibits thymidylate synthase, resulting in the depletion of thymidine triphosphate (TTP), one of the four nucleotide triphosphates used in the in vivo synthesis of DNA. In some aspects, other fluorouracil metabolites incorporate into both RNA and DNA; incorporation into RNA results in major effects on both RNA processing and functions. In some aspects, the dose of fluorouracil can be 500-3000 mg/m². In some aspects, the dose of fluorouracil can be 400 mg/m². In some aspects, the dose of fluorouracil can be 1200 mg/m². In some aspects, the dose of fluorouracil can be 2400 mg/m². In some aspects, the dose of fluorouracil can be dependent on whether administered as a bolus or an infusion. In some aspects, any known therapeutic dose of fluorouracil can be used.

In some aspects, irinotecan hydrochloride, or a derivative thereof, is administered to a subject or a population of cells. In some aspects, irinotecan hydrochloride is a hydrochloride salt of a semisynthetic derivative of camptothecin, a cytotoxic, quinoline-based alkaloid extracted from the Asian tree Camptotheca acuminate. In some aspects, irinotecan, a prodrug, can be converted to a biologically active metabolite 7-ethyl-10-hydroxy-camptothecin (SN-38) by a carboxylesterase-converting enzyme. One thousand-fold more potent than its parent compound irinotecan, SN-38 can inhibit topoisomerase I activity by stabilizing the cleavable complex between topoisomerase I and DNA, resulting in DNA breaks that inhibit DNA replication and trigger apoptotic cell death. Because ongoing DNA synthesis is necessary for irinotecan to exert its cytotoxic effects, it can be classified as an S-phase-specific agent. In some aspects, the alternatives to Irinotecan hydrochloride can be irinotecan. In some aspects, the dose of irinotecan hydrochloride can be 100-500 mg/m². In some aspects, the dose of irinotecan hydrochloride can be 180 mg/m². In some aspects, the dose of irinotecan hydrochloride can be 125 mg/m². In some aspects, any known therapeutic dose of irinotecan hydrochloride can be used.

In some aspects, oxaliplatin, or a derivative thereof, is administered to a subject or a population of cells. In some aspects, oxaliplatin can be an organoplatinum complex in which the platinum atom is complexed with 1,2-diaminocyclohexane (DACH) and with an oxalate ligand as a ‘leaving group.’ A ‘leaving group’ is an atom or a group of atoms that is displaced as a stable species taking with it the bonding electrons. In some aspects, after displacement of the labile oxalate ligand leaving group, active oxaliplatin derivatives, such as monoaquo and diaquo DACH platinum, alkylate macromolecules, forming both inter-and intra-strand platinum-DNA crosslinks, can result in inhibition of DNA replication and transcription and cell-cycle nonspecific cytotoxicity. In some aspects, the DACH side chain appears to inhibit alkylating-agent resistance. In some aspects, the dose of oxaliplatin can be 50-200 mg/m². In some aspects, the dose of oxaliplatin can be 85 mg/m². In some aspects, any known therapeutic dose of oxaliplatin can be used.

In some aspects, the combination treatment of leucovorin calcium, fluorouracil, irinotecane hydrochloride, and oxaliplatin is referred to as FOLFIRINOX. FOLFIRINOX contains the drugs leucovorin calcium (folinic acid), fluorouracil, irinotecan hydrochloride, and oxaliplatin. In some aspects, fluorouracil inhibits thymidylate synthase, impairs DNA replication, disrupts RNA processing. In some aspects, leucovorin enhances 5-fluorouracil uptake. In some aspects, irinotecane traps topoisomerase I, leading to single-strand DNA breaks. In some aspects, oxaliplatin induces DNA damage (interstrand cross-links, protein-DNA cross-links, single and double-strand breaks).

### D. Compositions

In some aspects, the disclosed compositions comprise one or more of the therapeutics disclosed herein.

In some aspects, the disclosed compositions comprise one or more of leucovorin calcium, fluorouracil, irinotecane hydrochloride, and oxaliplatin. In some aspects, the disclosed compositions comprise leucovorin calcium. In some aspects, the disclosed compositions comprise fluorouracil (5-FU). In some aspects, the disclosed compositions comprise irinotecan hydrochloride. In some aspects, the disclosed compositions comprise oxaliplatin.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The disclosed therapeutics (e.g., leucovorin calcium, fluorouracil, irinotecane hydrochloride, and oxaliplatin) can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed therapeutics (e.g., leucovorin calcium, fluorouracil, irinotecane hydrochloride, and oxaliplatin) described herein, or a pharmaceutically acceptable salt or solvate thereof, and a pharmaceutically acceptable carrier, buffer, or diluent. In various aspects, the peptide of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions, comprising the therapeutics disclosed herein, and a pharmaceutically acceptable carrier. In one aspect, disclosed are pharmaceutical compositions comprising the disclosed therapeutics. That is, a pharmaceutical composition can be provided comprising a therapeutically effective amount of at least one disclosed therapeutic and a pharmaceutically acceptable carrier.

In certain aspects, the disclosed pharmaceutical compositions comprise the disclosed therapeutics (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the therapeutics described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC: Cholesterol: peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed therapeutics in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a disclosed therapeutics (e.g., leucovorin calcium, fluorouracil, irinotecane hydrochloride, and oxaliplatin) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### E. Methods

Disclosed are methods of applying an alternating electric field and administering a combination of leucovorin calcium, fluorouracil, irinotecane hydrochloride, and oxaliplatin to the subject in need thereof and/or to a population of cells. In some aspects, the combination of leucovorin calcium, fluorouracil, irinotecane hydrochloride, and oxaliplatin is referred to as FOLFIRINOX.

In some aspects, any of the alternating electric fields disclosed herein can be applied to a subject and/or population of cells. For example, applying an alternating electric field can occur before, after, simultaneously, or a combination thereof, with the administration of leucovorin calcium, fluorouracil, irinotecane hydrochloride, and oxaliplatin. In some aspects, any of the frequencies and field strengths disclosed herein can be used. For example, in some aspects, the frequency of the alternating electric field is between 50 kHz and 1 MHz, preferably between 150 and 250 kHz. In some aspects, the alternating electric field has a field strength of 0.5 to 10 V/cm, preferably, 0.7 or 1 V/cm, in at least a portion of the target region. In some aspects, different frequencies and field strengths can be used throughout any of the disclosed methods. For example, any of the disclosed methods can include two or more of the disclosed treatment cycles. In some aspects, during a first treatment cycle a first frequency and/or field strength can be used and during a second treatment cycle a different frequency and/or field strength can be used.

In some aspects, any of the doses, treatment cycles, or dosing regimens of leucovorin calcium, fluorouracil, irinotecane hydrochloride, and oxaliplatin disclosed herein can be administered to a subject. In some aspects of the disclosed methods, the leucovorin calcium, fluorouracil, irinotecane hydrochloride, and oxaliplatin are administered at therapeutically effective doses.

### 1. Methods of Treating

Disclosed are method of treating a subject in need thereof comprising applying an alternating electric field, at a frequency for a period of time, to a target site of the subject in need thereof; and administering a combination of leucovorin calcium, fluorouracil, irinotecane hydrochloride, and oxaliplatin to the subject in need thereof.

In some aspects, the subject in need thereof has cancer. In some aspects, the cancer is pancreatic cancer, lung cancer, breast cancer, cervical cancer, colon cancer, gastric cancer, renal cancer, prostate cancer, thyroid cancer, glioblastoma, mesothelioma, non-small cell lung, ovarian cancer, hepatic cancer. Thus, for example, in some aspects, the subject has pancreatic cancer.

In some aspects, the target site comprises one or more cancer cells. In some aspects, the one or more cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

In some aspects, the cancer has metastasized.

In some aspects, the methods of treating cause one or more cancer cells to be killed. In some aspects, the one or more cancer cells that are killed are in or adjacent to the target site.

In some aspects, the cancer cells lack BRCA mutations. Thus, in some aspects, the subject has wild type BRCA genes. In some aspects, the cancer cells have normal, or wild type, BRCA-dependent DNA repair pathways.

In some aspects, the method of treating is a method of sensitizing BRCA wild-type pancreatic cancer cells to treatment with FOLFIRINOX. In some aspects, FOLFIRINOX is more effective in BRCA mutant cells. Thus, the ability to make BRCA wild type cells appear more like BRCA mutant cells would allow for FOLFIRINOX to be more effective. In some aspects, the alternating electric field reduces expression of the BRCA DNA damage repair pathway. In some aspects, this reduced expression of the BRCA DNA damage repair pathway allows for more effective treatment with FOLFIRINOX. Therefore, disclosed are method of treating a subject having cancer, wherein BRCA wild-type pancreatic cancer cells are sensitized to treatment with FOLFIRINOX by treatment with an alternating electric field.

In some aspects, the methods comprise first selecting or identifying a subject as having wild type BRCA genes.

In some aspects of the disclosed methods, cell volume is increased, clonogenicity is reduced, tumor volume is decreased, and/or a frequency of abnormal mitotic events in the cells are increased.

In some aspects of the disclosed methods, an inflammatory response is induced. Thus, in some aspects, the co-application of an alternating electric field and FOLIRINOX promotes an inflammatory response.

### 2. Methods of Reducing Viability of Cancer Cells

Disclosed are methods of reducing viability of cancer cells comprising applying an alternating electric field, at a frequency for a period of time, to a population of cancer cells; and contacting the population of cells with a combination of leucovorin calcium, fluorouracil, irinotecane hydrochloride, and oxaliplatin.

In some aspects, the population of cancer cells are in vitro. In some aspects, the population of cancer cells are in a subject. In some aspects, when the population of cancer cells are in a subject, contacting the population of cancer cells with a combination of leucovorin calcium, fluorouracil, irinotecane hydrochloride, and oxaliplatin comprises administering leucovorin calcium, fluorouracil, irinotecane hydrochloride, and oxaliplatin to a target site of the subject, wherein the target site comprises a population of cancer cells. Thus, disclosed are methods of reducing viability of cancer cells comprising applying an alternating electric field, at a frequency for a period of time, to a target site in a subject in need thereof, wherein the target site comprises cancer cells; and administering to the subject in need thereof a combination of leucovorin calcium, fluorouracil, irinotecane hydrochloride, and oxaliplatin.

In some aspects, the population of cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

In some aspects, the cancer cells lack BRCA mutations. Thus, in some aspects, the subject has wild type BRCA genes. In some aspects, the cancer cells have normal, or wild type, BRCA-dependent DNA repair pathways. In some aspects, the alternating electric field can sensitize the wild type BRCA cells to treatment with leucovorin calcium, fluorouracil, irinotecane hydrochloride, and oxaliplatin. In some aspects, the methods comprise first selecting or identifying a subject as having wild type BRCA genotype.

In some aspects of the disclosed methods, cell volume is increased, clonogenicity is reduced, tumor volume is decreased, and/or a frequency of abnormal mitotic events in the cells are increased.

In some aspects of the disclosed methods, an inflammatory response is induced. Thus, in some aspects, the co-application of an alternating electric field and FOLIRINOX promotes an inflammatory response.

### 3. Methods of Increasing Apoptosis of Cancer Cells

Disclosed are methods of increasing apoptosis of cancer cells comprising applying an alternating electric field, at a frequency for a period of time, to a population of cancer cells; and contacting the population of cancer cells with a combination of leucovorin calcium, fluorouracil, irinotecane hydrochloride, and oxaliplatin.

In some aspects, the population of cancer cells are in vitro. In some aspects, the population of cancer cells are in a subject. In some aspects, when the population of cancer cells are in a subject, contacting the population of cancer cells with a combination of leucovorin calcium, fluorouracil, irinotecane hydrochloride, and oxaliplatin comprises administering leucovorin calcium, fluorouracil, irinotecane hydrochloride, and oxaliplatin to a target site of the subject, wherein the target site comprises a population of cancer cells. Thus, disclosed are methods of increasing apoptosis of cancer cells comprising applying an alternating electric field, at a frequency for a period of time, to a target site in a subject in need thereof, wherein the target site comprises cancer cells; and administering to the subject in need thereof a combination of leucovorin calcium, fluorouracil, irinotecane hydrochloride, and oxaliplatin.

In some aspects, the population of cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

In some aspects, the cancer cells lack BRCA mutations. Thus, in some aspects, the subject has wild type BRCA genes. In some aspects, the cancer cells have normal, or wild type, BRCA-dependent DNA repair pathways. In some aspects, the alternating electric field can sensitize the wild type BRCA cells to treatment with leucovorin calcium, fluorouracil, irinotecane hydrochloride, and oxaliplatin. In some aspects, the methods comprise first selecting or identifying a subject as having wild type BRCA genes.

In some aspects of the disclosed methods, cell volume is increased, clonogenicity is reduced, tumor volume is decreased, and/or a frequency of abnormal mitotic events in the cells are increased.

In some aspects of the disclosed methods, an inflammatory response is induced. Thus, in some aspects, the co-application of an alternating electric field and FOLIRINOX promotes an inflammatory response.

### 4. Methods of Reducing the Number of Cancer Cells

Disclosed are methods of reducing the number of cancer cells comprising applying an alternating electric field, at a frequency for a period of time, to a population of cancer cells; and contacting the population of cancer cells with a combination of leucovorin calcium, fluorouracil, irinotecane hydrochloride, and oxaliplatin.

In some aspects, the population of cancer cells are in vitro. In some aspects, the population of cancer cells are in a subject. In some aspects, when the population of cancer cells are in a subject, contacting the population of cancer cells with a combination of leucovorin calcium, fluorouracil, irinotecane hydrochloride, and oxaliplatin comprises administering leucovorin calcium, fluorouracil, irinotecane hydrochloride, and oxaliplatin to a target site of the subject, wherein the target site comprises a population of cancer cells. Thus, disclosed are methods of reducing the number of cancer cells comprising applying an alternating electric field, at a frequency for a period of time, to a target site in a subject in need thereof, wherein the target site comprises cancer cells; and administering to the subject in need thereof a combination of leucovorin calcium, fluorouracil, irinotecane hydrochloride, and oxaliplatin.

In some aspects, the population of cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

In some aspects, the cancer cells lack BRCA mutations. Thus, in some aspects, the subject has wild type BRCA genes. In some aspects, the cancer cells have normal, or wild type, BRCA-dependent DNA repair pathways. In some aspects, the alternating electric field can sensitize the wild type BRCA cells to treatment with leucovorin calcium, fluorouracil, irinotecane hydrochloride, and oxaliplatin. In some aspects, the methods comprise first selecting or identifying a subject as having wild type BRCA genes.

In some aspects of the disclosed methods, cell volume is increased, clonogenicity is reduced, tumor volume is decreased, and/or a frequency of abnormal mitotic events in the cells are increased.

In some aspects of the disclosed methods, an inflammatory response is induced. Thus, in some aspects, the co-application of an alternating electric field and FOLIRINOX promotes an inflammatory response.

### 5. Methods of Sensitizing BRCA Wild-Type Cancer Cells to Treatment with FOLFIRINOX

Disclosed are methods of sensitizing BRCA wild-type cancer cells to treatment with FOLFIRINOX comprising applying an alternating electric field, at a frequency for a period of time, to a population of cancer cells; and contacting the population of cancer cells with a combination of leucovorin calcium, fluorouracil, irinotecane hydrochloride, and oxaliplatin.

In some aspects, FOLFIRINOX is more effective in BRCA mutant cells. Thus, the ability to make BRCA wild type cells appear more like BRCA mutant cells allows for FOLFIRINOX to be more effective. In some aspects, the alternating electric field reduces expression of the BRCA DNA damage repair pathway. In some aspects, this reduced expression of the BRCA DNA damage repair pathway sensitizes the cells to treatment with FOLFIRINOX.

In some aspects, the population of cancer cells are in vitro. In some aspects, the population of cancer cells are in a subject. Thus, when the population of cancer cells are in a subject, contacting the population of cancer cells with a combination of leucovorin calcium, fluorouracil, irinotecane hydrochloride, and oxaliplatin comprises administering leucovorin calcium, fluorouracil, irinotecane hydrochloride, and oxaliplatin to a target site of the subject, wherein the target site comprises a population of cancer cells. Thus, disclosed are methods of sensitizing BRCA wild-type cancer cells to treatment with FOLFIRINOX comprising applying an alternating electric field, at a frequency for a period of time, to a target site in a BRCA wild type subject, wherein the target site comprises cancer cells; and administering to the subject in need thereof a combination of leucovorin calcium, fluorouracil, irinotecane hydrochloride, and oxaliplatin.

In some aspects, the population of cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

In some aspects of the disclosed methods, cell volume is increased, clonogenicity is reduced, tumor volume is decreased, and/or a frequency of abnormal mitotic events in the cells are increased.

In some aspects of the disclosed methods, an inflammatory response is induced. Thus, in some aspects, the co-application of an alternating electric field and FOLIRINOX promotes an inflammatory response.

### F. Dosing Regimen

Disclosed are dosing regimens comprising applying an alternating electric field to a target site of a subject in need thereof; and administering a combination of leucovorin calcium, fluorouracil, irinotecane hydrochloride, and oxaliplatin to the subject in need thereof. In some aspects, any of the doses of leucovorin calcium, fluorouracil, irinotecane hydrochloride, and oxaliplatin described herein can be used. In some aspects, any of the frequency or field strengths described herein for the alternating electric fields can be used.

In some aspect, a dosing regimen can comprise at least one treatment cycle comprising both alternating electric fields and leucovorin calcium, fluorouracil, irinotecane hydrochloride, and oxaliplatin. In some aspects, a dosing regimen can comprise at least 1, 2, 3, 4, 5, 6, 7, 8, 9, 10, 11, or 12 treatment cycles.

In some aspects, administering a combination of leucovorin calcium, fluorouracil, irinotecane hydrochloride, and oxaliplatin can refer to administering all four of leucovorin calcium, fluorouracil, irinotecane hydrochloride, and oxaliplatin but each one can be administered in a specific amount and at a specific time. In some aspects, one or more of leucovorin calcium, fluorouracil, irinotecane hydrochloride, and oxaliplatin are not administered simultaneously.

In some aspects, administering a combination of leucovorin calcium, fluorouracil, irinotecane hydrochloride, and oxaliplatin can mean administering each of leucovorin calcium, fluorouracil, irinotecane hydrochloride, and oxaliplatin in a single treatment cycle.

In some aspects, a dosing regimen comprises at least one treatment cycle of an effective amount of an alternating electric field and any of the combinations of leucovorin calcium, fluorouracil, irinotecane hydrochloride, and oxaliplatin disclosed herein. In some aspects, a treatment cycle can be followed by a rest phase. In some aspects, the rest phase of a dosing regimen can be a period of time where none of leucovorin calcium, fluorouracil, irinotecane hydrochloride, and oxaliplatin are administered. In some aspects, the rest phase of a dosing regimen can be a period of time where no alternating electric fields are applied. In some aspects, the rest phase of a dosing regimen can be a period of time where no alternating electric fields are applied and none of leucovorin calcium, fluorouracil, irinotecane hydrochloride, and oxaliplatin are administered.

In some aspects, the order of administration of leucovorin calcium, fluorouracil, irinotecane hydrochloride, and oxaliplatin can vary. In some aspects, oxaliplatin can be administered first, followed by leucovorin, followed by irinotecan, and then fluorouracil. In some aspects, there is no overlap being dosing of each of leucovorin calcium, fluorouracil, irinotecane hydrochloride, and oxaliplatin. In some aspects, two or more of leucovorin calcium, fluorouracil, irinotecane hydrochloride, and oxaliplatin can be administered simultaneously.

In some aspects, a single treatment cycle comprises each of leucovorin calcium, fluorouracil, irinotecane hydrochloride, and oxaliplatin being administered in the same day. In some aspects, a single treatment cycle comprises each of leucovorin calcium, fluorouracil, irinotecane hydrochloride, and oxaliplatin being administered within 1, 2, 3, 4, 5, 6, 7, 8, 9, 10, 11, 12, 13, or 14 days of each other.

In some aspects, FOLFIRINOX treatment comprises oxaliplatin intravenous (I.V.) infusion given over 2 hours on Day 1; folinic acid (Leucovorin) I.V. infusion given over 2 hours on Day 1; irinotecan I.V. infusion given over 90 minutes on Day 1; fluorouracil I.V. push given over 3 to 5 minutes on Day 1; and fluorouracil continuous I.V. infusion given via infusion pump over 46 hours beginning Day 1. Thus, in some aspects, a single treatment cycle of FOLFIRINOX can comprise two separate doses of fluorouracil.

In some aspects, a single treatment cycle of FOLFIRINOX can take about 2 days. In some aspects, each two-day FOLFIRINOX treatment (single treatment cycle) is repeated every 14 days. In some aspects, at least 1, 2, 3, 4, 5, 6, 7, 8, 9, 10, 11, or 12 treatment cycles can be administered to a subject.

In some aspects, a treatment cycle of FOLFIRINOX comprises oxaliplatin at a dose of 85 mg per square meter, given as a 2-hour intravenous infusion, immediately followed by leucovorin at a dose of 400 mg per square meter, given as a 2-hour intravenous infusion, with the addition, after 30 minutes, of irinotecan at a dose of 180 mg per square meter, given as a 90-minute intravenous infusion. This treatment was immediately followed by fluorouracil at a dose of 400 mg per square meter, administered by intravenous bolus, followed by a continuous intravenous infusion of 2400 mg per square meter over a 46-hour period every 2 weeks.

In some aspects, a treatment cycle of FOLFIRINOX comprises fluorouracil administered as a bolus of 400 mg/m², a bolus of leucovorin at 400 mg/m², followed by continuous infusion of fluorouracil at 1200 mg/m² per day for 46 hours. In some aspects, oxaliplatin is administered, prior to the continuous fluorouracil infusion, at a dose of 85 mg/m², and irinotecan is administered, prior to the continuous fluorouracil infusion, at a dose of 180 mg/m².

In some aspects, the alternating electric field is applied before, after, or simultaneously with administering the combination of leucovorin calcium, fluorouracil, irinotecane hydrochloride, and oxaliplatin. In some aspects, each treatment cycle comprises not only the administration of leucovorin calcium, fluorouracil, irinotecane hydrochloride, and oxaliplatin but also applying an alternating electric field. In some aspects, an alternating electric field is applied continuously during the administration of leucovorin calcium, fluorouracil, irinotecane hydrochloride, and oxaliplatin.

In some aspects, at least one treatment cycle comprises at least a portion of applying an alternating electric field is performed after the administering a combination of leucovorin calcium, fluorouracil, irinotecane hydrochloride, and oxaliplatin.

In some aspects, at least one treatment cycle comprises at least a portion of applying an alternating electric field is performed before the administering a combination of leucovorin calcium, fluorouracil, irinotecane hydrochloride, and oxaliplatin.

In some aspects, at least one treatment cycle comprises at least a portion of applying the alternating electric field is performed simultaneously with at least a portion of the administering the combination of leucovorin calcium, fluorouracil, irinotecane hydrochloride, and oxaliplatin.

In some aspects, at least one treatment cycle comprises at least a portion of applying an alternating electric field is performed before the leucovorin calcium, fluorouracil, irinotecane hydrochloride, and oxaliplatin are eliminated from the subject's body. Thus, in some aspects, each of leucovorin calcium, fluorouracil, irinotecane hydrochloride, and oxaliplatin are present in a subject's body while the subject receives an alternating electric field.

In some aspects, leucovorin calcium, fluorouracil, irinotecane hydrochloride, and oxaliplatin can be administered using any known technique. For example, one or more of leucovorin calcium, fluorouracil, irinotecane hydrochloride, and oxaliplatin can be administered using intravenous administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. In some aspects, each of leucovorin calcium, fluorouracil, irinotecane hydrochloride, and oxaliplatin are administered via the same route. In some aspects, one or more of leucovorin calcium, fluorouracil, irinotecane hydrochloride, and oxaliplatin are administered intravenously.

In some instances, the administration of the fluorouracil is accomplished by direct administration of the fluorouracil to the subject. In some instances, the administration of the fluorouracil is accomplished by indirect administration of the fluorouracil to the subject. The indirect administration of the fluorouracil can be accomplished, for example, by administering a pre-prodrug (e.g., capecitabine) to the subject, wherein the pre-prodrug is converted to fluorouracil in the subject's body. In some aspects, any of leucovorin calcium, fluorouracil, irinotecane hydrochloride, and oxaliplatin can be administered using indirect administration.

The term “indirectly” or “indirect,” as used herein, refers to administering a precursor, pre-prodrug, prodrug, or a different drug to a subject that is converted to fluorouracil in the body (e.g., the liver). See, e.g., Schellens, The Oncologist, 2007; 12:152-15; Hanneke et al., Cancer Research, 63, 7609-7612, Nov. 15, 2003. In some instances, administering a precursor, pre-prodrug, prodrug, or a different drug to a subject can be advantageous by reducing side effects or altering pharmacokinetics in a desirable manner.

In some aspects, each dosing regimen, or treatment cycle, comprises applying an alternating electric field. In some aspects, applying an alternating electric field has a duration of at least 12, 24, 36, 48, 60 or 72 hours. In some aspects, applying an alternating electric field has a duration of at least 1, 2, 3, 4, 5, 6, 7, 8, 9, 10, 11, 12, 13, or 14 days. In some aspects, the alternating electric field can be continuous or intermittent. For example, applying an alternating electric field for 72 hours can be continuous for 72 hours or can be an intermittent cycle throughout the 72 hours.

In some aspects, applying an alternating electric field at least before administering one or more of leucovorin calcium, fluorouracil, irinotecane hydrochloride, and oxaliplatin allows the alternating electric field to downregulate DNA damage repair pathways that are required for the repair of the damage induced by the treatment of leucovorin calcium, fluorouracil, irinotecane hydrochloride, and oxaliplatin. In some aspects, an alternating electric field can be applied at least 12, 24, 48 or 72 hours prior to administration of one or more of leucovorin calcium, fluorouracil, irinotecane hydrochloride, and oxaliplatin. In some aspects, an alternating electric field is applied before, during, and/or after administration of one or more of leucovorin calcium, fluorouracil, irinotecane hydrochloride, and oxaliplatin.

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of leucovorin calcium, fluorouracil, irinotecane hydrochloride, and oxaliplatin and one or more materials for delivering alternating electric fields, such as the Optune system. For example disclosed are kits comprising each of leucovorin calcium, fluorouracil, irinotecane hydrochloride, and oxaliplatin and one or more materials for delivering alternating electric fields, such as the Optune system.

### H. Illustrative Embodiments

Embodiment 1: A method of treating a subject in need thereof comprising: applying an alternating electric field, at a frequency for a period of time, to a target site of the subject in need thereof; and administering a combination of Leucovorin calcium, Fluorouracil, Irinotecane hydrochloride, Oxaliplatin to the subject in need thereof.

Embodiment 2: The method of embodiment 1, wherein the combination of Leucovorin calcium, Fluorouracil, Irinotecane hydrochloride, Oxaliplatin is FOLFIRINOX.

Embodiment 3: The method of any one of embodiments 1-2, wherein the subject has cancer.

Embodiment 4: The method of embodiment 3, wherein the cancer is pancreatic cancer, lung cancer, breast cancer, cervical cancer, colon cancer, gastric cancer, renal cancer, prostate cancer, thyroid cancer, glioblastoma, mesothelioma, non-small cell lung, ovarian cancer, hepatic cancer.

Embodiment 5: The method of any one of embodiments 1-4, wherein the target site comprises one or more cancer cells.

Embodiment 6: The method of embodiment 5, wherein the one or more cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

Embodiment 7: The method of any one of embodiments 3-6, wherein the cancer has metastasized.

Embodiment 8: The method of any one of embodiments 1-7, wherein one or more cancer cells are killed.

Embodiment 9: A method of reducing viability of cancer cells comprising:
applying an alternating electric field, at a frequency for a period of time, to a population of cancer cells in need thereof; and contacting the population of cells with a combination of Leucovorin calcium, Fluorouracil, Irinotecane hydrochloride, Oxaliplatin.

Embodiment 10: A method of increasing apoptosis of cancer cells comprising:
applying an alternating electric field, at a frequency for a period of time, to a population of cancer cells; and contacting the population of cancer cells with a combination of Leucovorin calcium, Fluorouracil, Irinotecane hydrochloride, Oxaliplatin.

Embodiment 11: A method of reducing the number of cancer cells comprising:
applying an alternating electric field, at a frequency for a period of time, to a population of cancer cells; and contacting the population of cancer cells with a combination of Leucovorin calcium, Fluorouracil, Irinotecane hydrochloride, Oxaliplatin.

Embodiment 12: The method of any one of embodiments 9-11, wherein the population of cells are in vitro.

Embodiment 13: The method of any one of embodiments 9-11, wherein the population of cells are in a subject

Embodiment 14: The method of any one of embodiments 9-13, wherein the population of cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

Embodiment 15: The method of any one of embodiments 5-14, wherein the cancer cells lack BRCA mutations.

Embodiment 16: The method of any one of embodiments 1-15, wherein the alternating electric field reduces expression of the BRCA DNA damage repair pathway.

Embodiment 17: The method of any one of embodiments 1-16, wherein the alternating electric field is applied before, after, or simultaneously with administering the combination of Leucovorin calcium, Fluorouracil, Irinotecane hydrochloride, Oxaliplatin.

Embodiment 18: The method of any one of embodiments 1-17, wherein at least a portion of the applying an alternating electric field is performed after the administering a combination of Leucovorin calcium, Fluorouracil, Irinotecane hydrochloride, and Oxaliplatin.

Embodiment 19: The method of any one of embodiments 1-18, wherein at least a portion of the applying an alternating electric field is performed before the administering a combination of Leucovorin calcium, Fluorouracil, Irinotecane hydrochloride, and Oxaliplatin.

Embodiment 20: The method of any one of embodiments 1-19, wherein at least a portion of the applying the alternating electric field is performed simultaneously with at least a portion of the administering the combination of Leucovorin calcium, Fluorouracil, Irinotecane hydrochloride, and Oxaliplatin.

Embodiment 21: The method of any one of embodiments 1-20, wherein at least a portion of the applying an alternating electric field is performed before the Leucovorin calcium, Fluorouracil, Irinotecane hydrochloride, and Oxaliplatin are eliminated from the subject's body.

Embodiment 22: The method of any one of claims 1-21, wherein the applying an alternating electric field has a duration of at least 12, 24, 36, 48, 60 or 72 hours.

Embodiment 23: The method of any one of embodiments 1-22, wherein step b) is performed 1, 2, 3, 4, 5, 6, 7, 8, 9, or 10 days after step a) is performed.

Embodiment 24: The method of any one of embodiments 1-23, wherein the frequency of the alternating electric field is between 50 kHz and 1 MHz.

Embodiment 25: The method of embodiment 24, wherein the frequency of the alternating electric field is between 150 and 250 kHz.

Embodiment 26: The method of any one of embodiments 1-25, wherein the alternating electric field has a field strength of 0.5 to 10 V/cm in at least a portion of the target region.

Embodiment 27: The method of any one of embodiments 1-26, wherein the Leucovorin calcium, Fluorouracil, Irinotecane hydrochloride, and Oxaliplatin are administered at therapeutically effective doses.

Embodiment 28: The method of any one of embodiments 1-27, wherein one or more of the combination of Leucovorin calcium, Fluorouracil, Irinotecane hydrochloride, and Oxaliplatin is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

Embodiment 29: The method of any one of embodiments 1-28, wherein the combination of Leucovorin calcium, Fluorouracil, Irinotecane hydrochloride, and Oxaliplatin is administered in a dosing regimen comprising administering oxaliplatin at a dose of 85 mg per square meter, given as a 2-hour intravenous infusion, immediately followed by leucovorin at a dose of 400 mg per square meter, given as a 2-hour intravenous infusion, and, after 30 minutes, administering irinotecan at a dose of 180 mg per square meter, given as a 90-minute intravenous infusion, and immediately followed by administering fluorouracil at a dose of 400 mg per square meter, by intravenous bolus, followed by a continuous intravenous infusion of 2400 mg per square meter of fluorouracil over a 46-hour period every 2 weeks.

Embodiment 30: The method of any one of embodiments 1-29, wherein cell volume is increased, clonogenicity is reduced, tumor volume is decreased, and/or the frequency of abnormal mitotic events in the cells are increased.

Embodiment 31: The method of any one of embodiments 1-30, wherein an inflammatory response is induced in the subject.

Embodiment 32: A combination of Leucovorin calcium, Fluorouracil, Irinotecane hydrochloride, Oxaliplatin for use in a method of treating a subject in need thereof comprising applying an alternating electric field, at a frequency for a period of time, to a target site of the subject in need thereof.

Embodiment 33: The combination of Leucovorin calcium, Fluorouracil, Irinotecane hydrochloride, Oxaliplatin of embodiment 32, wherein the combination of Leucovorin calcium, Fluorouracil, Irinotecane hydrochloride, Oxaliplatin is FOLFIRINOX.

Embodiment 34: The combination of Leucovorin calcium, Fluorouracil, Irinotecane hydrochloride, Oxaliplatin of any one of embodiments 32-33, wherein the subject has cancer.

Embodiment 35: The combination of Leucovorin calcium, Fluorouracil, Irinotecane hydrochloride, Oxaliplatin of embodiment 34, wherein the cancer is pancreatic cancer, lung cancer, breast cancer, cervical cancer, colon cancer, gastric cancer, renal cancer, prostate cancer, thyroid cancer, glioblastoma, mesothelioma, non-small cell lung, ovarian cancer, hepatic cancer.

Embodiment 36: The combination of Leucovorin calcium, Fluorouracil, Irinotecane hydrochloride, Oxaliplatin of any one of embodiments 32-35, wherein the target site comprises one or more cancer cells.

Embodiment 37: The combination of Leucovorin calcium, Fluorouracil, Irinotecane hydrochloride, Oxaliplatin of embodiment 36, wherein the one or more cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

Embodiment 38: The combination of Leucovorin calcium, Fluorouracil, Irinotecane hydrochloride, Oxaliplatin of any one of embodiments 32-37, wherein the cancer has metastasized.

Embodiment 39: The combination of Leucovorin calcium, Fluorouracil, Irinotecane hydrochloride, Oxaliplatin of any one of embodiments 32-38, wherein one or more cancer cells are killed.

Embodiment 40: A combination of Leucovorin calcium, Fluorouracil, Irinotecane hydrochloride, Oxaliplatin for use in reducing viability of cancer cells comprising applying an alternating electric field, at a frequency for a period of time, to a population of cancer cells in need thereof.

Embodiment 41: A combination of Leucovorin calcium, Fluorouracil, Irinotecane hydrochloride, Oxaliplatin for use in increasing apoptosis of cancer cells comprising: applying an alternating electric field, at a frequency for a period of time, to a population of cancer cells.

Embodiment 42: A combination of Leucovorin calcium, Fluorouracil, Irinotecane hydrochloride, Oxaliplatin for use in reducing the number of cancer cells comprising: applying an alternating electric field, at a frequency for a period of time, to a population of cancer cells.

Embodiment 43: The combination of Leucovorin calcium, Fluorouracil, Irinotecane hydrochloride, Oxaliplatin of any one of embodiments 40-42, wherein the population of cells are in vitro.

Embodiment 44: The combination of Leucovorin calcium, Fluorouracil, Irinotecane hydrochloride, Oxaliplatin of any one of embodiments 40-43, wherein the population of cells are in a subject.

Embodiment 45: The combination of Leucovorin calcium, Fluorouracil, Irinotecane hydrochloride, Oxaliplatin of any one of embodiments 40-44, wherein the population of cancer cells are pancreatic cancer cells, lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, non-small cell lung cancer cells, ovarian cancer cells, hepatic cancer cells.

Embodiment 46: The combination of Leucovorin calcium, Fluorouracil, Irinotecane hydrochloride, Oxaliplatin of any one of embodiments 36-45, wherein the cancer cells lack BRCA mutations.

Embodiment 47: The combination of Leucovorin calcium, Fluorouracil, Irinotecane hydrochloride, Oxaliplatin of any one of embodiments 32-46, wherein the alternating electric field reduces expression of the BRCA DNA damage repair pathway.

Embodiment 48: The combination of Leucovorin calcium, Fluorouracil, Irinotecane hydrochloride, Oxaliplatin of any one of embodiments 32-47, wherein the alternating electric field is applied before, after, or simultaneously with administering the combination of Leucovorin calcium, Fluorouracil, Irinotecane hydrochloride, Oxaliplatin.

Embodiment 49: The combination of Leucovorin calcium, Fluorouracil, Irinotecane hydrochloride, Oxaliplatin of any one of embodiments 32-48, wherein at least a portion of the applying an alternating electric field is performed after the administering a combination of Leucovorin calcium, Fluorouracil, Irinotecane hydrochloride, and Oxaliplatin.

Embodiment 50: The combination of Leucovorin calcium, Fluorouracil, Irinotecane hydrochloride, Oxaliplatin of any one of embodiments 32-49, wherein at least a portion of the applying an alternating electric field is performed before the administering a combination of Leucovorin calcium, Fluorouracil, Irinotecane hydrochloride, and Oxaliplatin.

Embodiment 51: The combination of Leucovorin calcium, Fluorouracil, Irinotecane hydrochloride, Oxaliplatin of any one of embodiments 32-50, wherein at least a portion of the applying the alternating electric field is performed simultaneously with at least a portion of the administering the combination of Leucovorin calcium, Fluorouracil, Irinotecane hydrochloride, and Oxaliplatin.

Embodiment 52: The combination of Leucovorin calcium, Fluorouracil, Irinotecane hydrochloride, Oxaliplatin of any one of embodiments 32-51, wherein at least a portion of the applying an alternating electric field is performed before the Leucovorin calcium, Fluorouracil, Irinotecane hydrochloride, and Oxaliplatin are eliminated from the subject's body.

Embodiment 53: The combination of Leucovorin calcium, Fluorouracil, Irinotecane hydrochloride, Oxaliplatin of any one of embodiments 32-52, wherein the applying an alternating electric field has a duration of at least 12, 24, 36, 48, 60 or 72 hours.

Embodiment 54: The combination of Leucovorin calcium, Fluorouracil, Irinotecane hydrochloride, Oxaliplatin of any one of embodiments 32-53, wherein step b) is performed 1, 2, 3, 4, 5, 6, 7, 8, 9, or 10 days after step a) is performed.

Embodiment 55: The combination of Leucovorin calcium, Fluorouracil, Irinotecane hydrochloride, Oxaliplatin of any one of embodiments 32-54, wherein the frequency of the alternating electric field is between 50 kHz and 1 MHz.

Embodiment 56: The combination of Leucovorin calcium, Fluorouracil, Irinotecane hydrochloride, Oxaliplatin of embodiment 55, wherein the frequency of the alternating electric field is between 150 and 250 kHz.

Embodiment 57: The combination of Leucovorin calcium, Fluorouracil, Irinotecane hydrochloride, Oxaliplatin of any one of embodiments 32-56, wherein the alternating electric field has a field strength of 0.5 to 10 V/cm in at least a portion of the target region.

Embodiment 58: The combination of Leucovorin calcium, Fluorouracil, Irinotecane hydrochloride, Oxaliplatin of any one of embodiments 32-57, wherein the Leucovorin calcium, Fluorouracil, Irinotecane hydrochloride, and Oxaliplatin are administered at therapeutically effective doses.

Embodiment 59: The combination of Leucovorin calcium, Fluorouracil, Irinotecane hydrochloride, Oxaliplatin of any one of embodiments 32-58, wherein one or more of the combination of Leucovorin calcium, Fluorouracil, Irinotecane hydrochloride, and Oxaliplatin is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

Embodiment 60: The combination of Leucovorin calcium, Fluorouracil, Irinotecane hydrochloride, Oxaliplatin of any one of embodiments 32-59, wherein the combination of Leucovorin calcium, Fluorouracil, Irinotecane hydrochloride, and Oxaliplatin is administered in a dosing regimen comprising administering oxaliplatin at a dose of 85 mg per square meter, given as a 2-hour intravenous infusion, immediately followed by leucovorin at a dose of 400 mg per square meter, given as a 2-hour intravenous infusion, and, after 30 minutes, administering irinotecan at a dose of 180 mg per square meter, given as a 90-minute intravenous infusion, and immediately followed by administering fluorouracil at a dose of 400 mg per square meter, by intravenous bolus, followed by a continuous intravenous infusion of 2400 mg per square meter of fluorouracil over a 46-hour period every 2 weeks.

Embodiment 61: The combination of Leucovorin calcium, Fluorouracil, Irinotecane hydrochloride, Oxaliplatin of any one of embodiments 32-60, wherein cell volume is increased, clonogenicity is reduced, tumor volume is decreased, and/or the frequency of abnormal mitotic events in the cells are increased.

Embodiment 62: The combination of Leucovorin calcium, Fluorouracil, Irinotecane hydrochloride, Oxaliplatin of any one of embodiments 32-61, wherein an inflammatory response is induced in the subject.

### EXAMPLES

### A. Tumor Treating Fields (TTFields) Sensitize BRCA Wild Type Pancreatic Cancer Cells to Treatment with FOLFIRINOX

### 1. Background

FOLFIRINOX is a first-line chemotherapy treatment for patients with advanced pancreatic cancer and good performance status. This combination regimen of fluorouracil, oxaliplatin, irinotecan and leucovorinn seems to be more effective in BRCA-mutated tumors due to the platinum-based component, acting by the induction of DNA damage. Tumor Treating Fields (TTFields) are electric fields that disrupt cellular processes crucial for cancer cell viability. TTFields have shown efficacy in preclinical pancreatic cancer models, and to reduce expression of proteins from the BRCA-dependent DNA repair pathway in various cancer types. The current study examined the use of TTFields to sensitize BRCA wild-type pancreatic cancer cells to treatment with FOLFIRINOX.

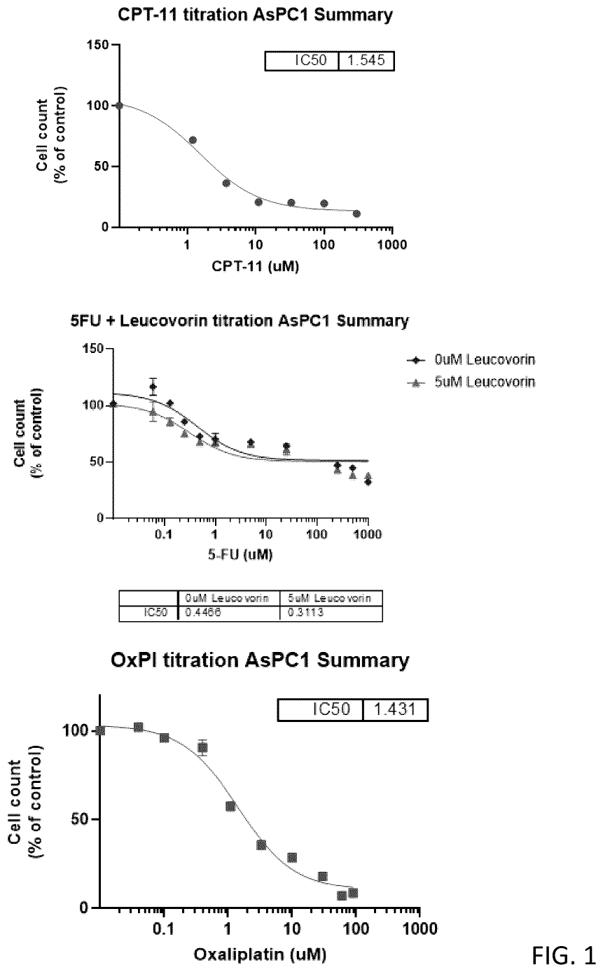

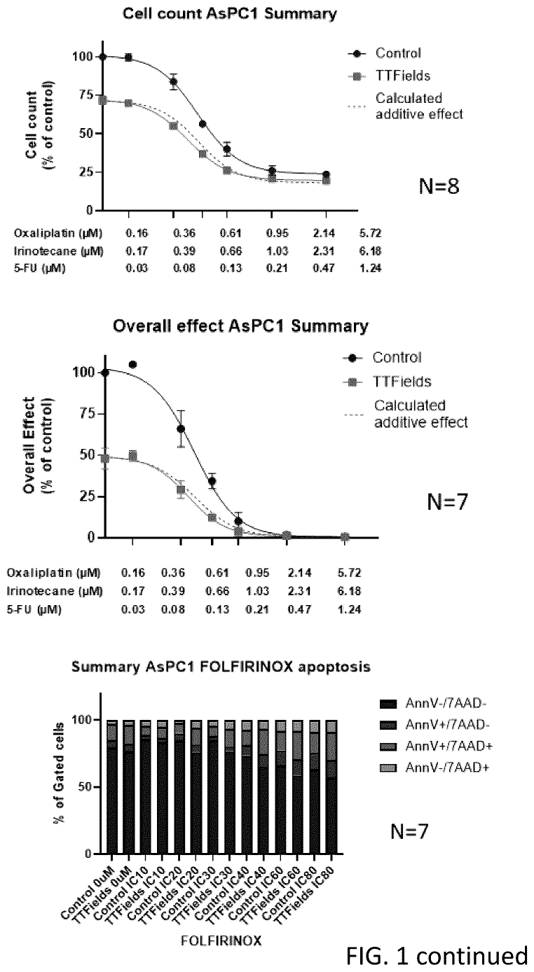

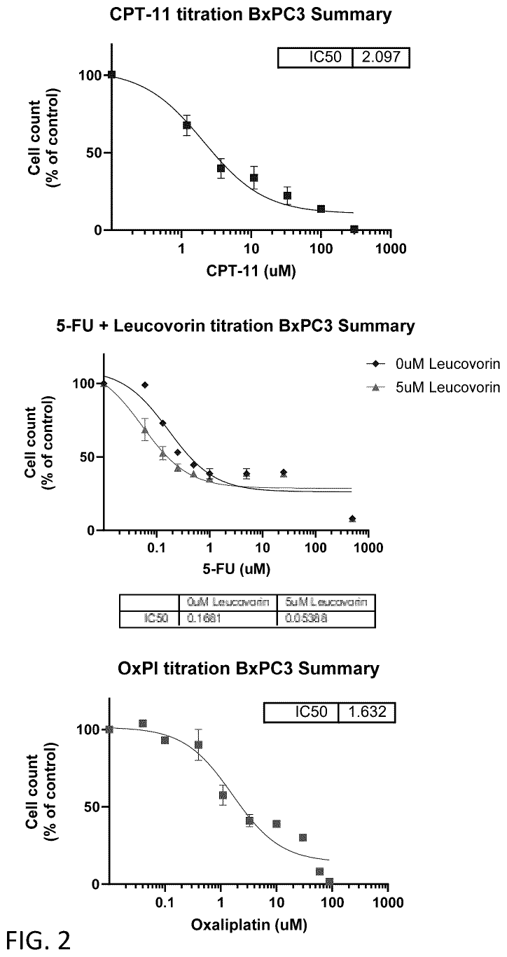

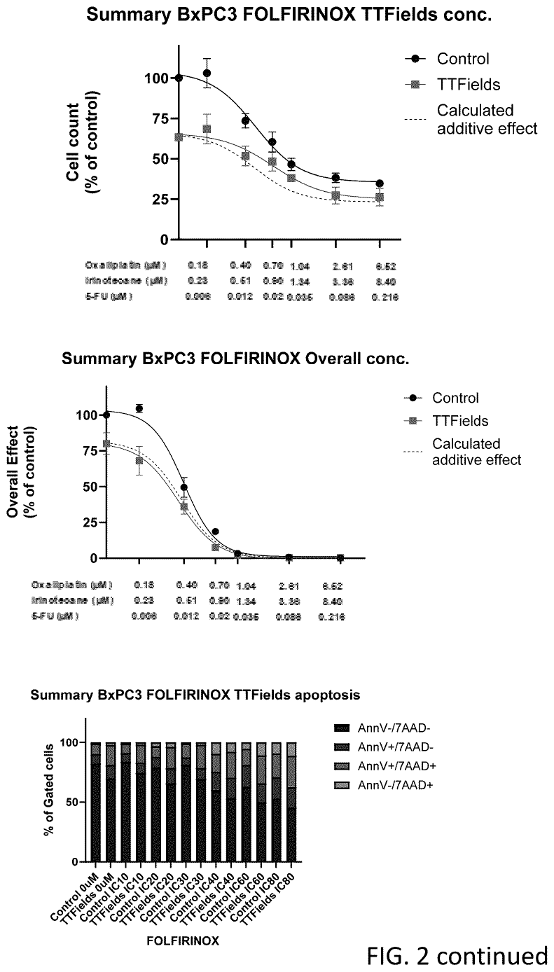

The below methods and results describe FIGS. 1 and 2.

### 2. Methods

Human pancreatic BRCA wild-type cancer cells BxPC3 and AsPC1 were treated with TTFields, using the inovitro device (Frequency: 150 kHz; Intensity: 0.7 V/cm RMS for BxPC3 cells; 1 V/cm RMS for AsPC1 cells). FOLFIRINOX was administered to the cells at increasing concentrations, with or without co-treatment with TTFields. After 72 h of treatment, cell count, colony formation, and apoptosis were measured. For mechanistic insight, RNA and protein were isolated from TTFields-treated and control samples following 24-and 48-hours treatment, and examined by RNA-sequencing, real-time PCR and Western blot.

### 3. Results:

TTFields application to the pancreatic cells together with FOLFIRINOX reduced cell count and colony formation and induced apoptosis relative to FOLFIRINOX or TTFields alone. TTFields downregulated DNA damage repair, DNA replication, and cell cycle related processes, with reduced expression of several central players in the BRCA DNA damage repair pathway.

### 4. Conclusions

Application of TTFields together with FOLFIRINOX exhibited improvement relative to FOLFIRINOX alone in treatment of pancreatic cancer cells lacking background BRCA mutations.

### B. the Effect of Ttfields and Folfirinox on Brca Wild Type Pancreatic Cancer Cells

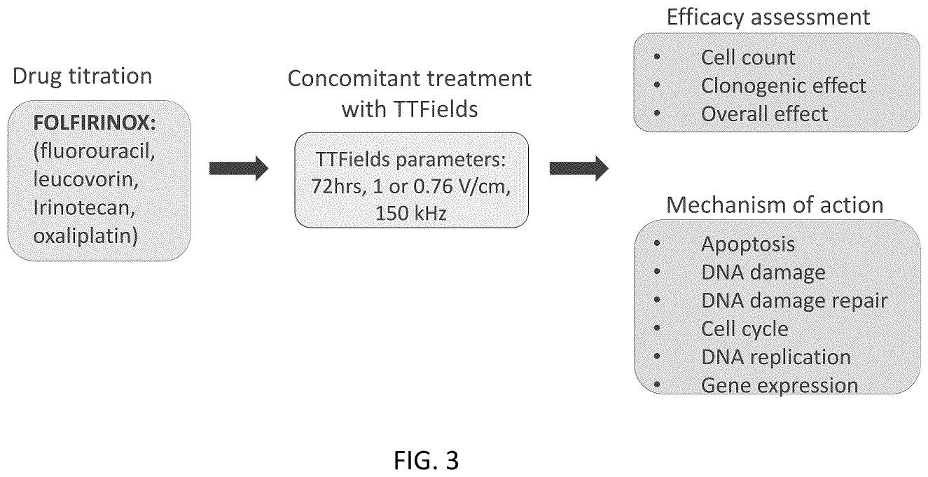

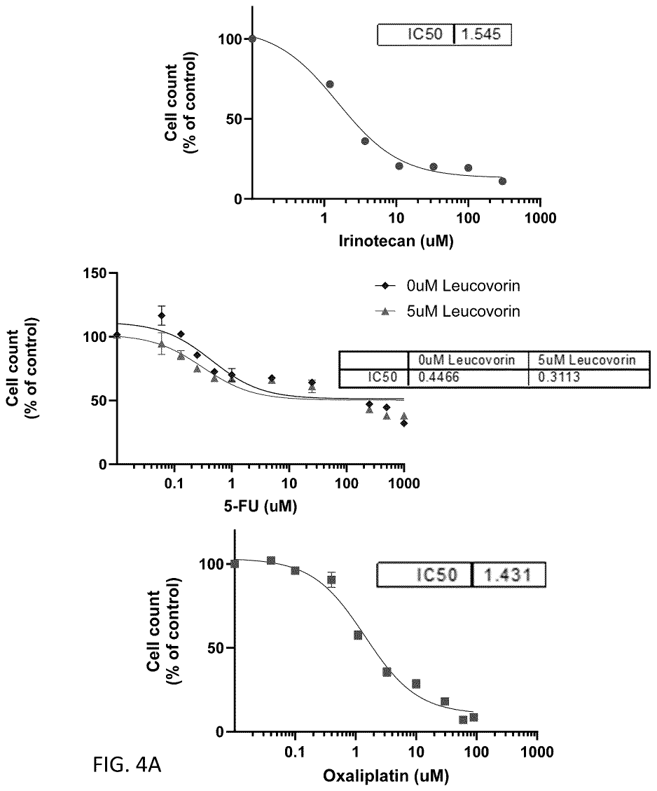

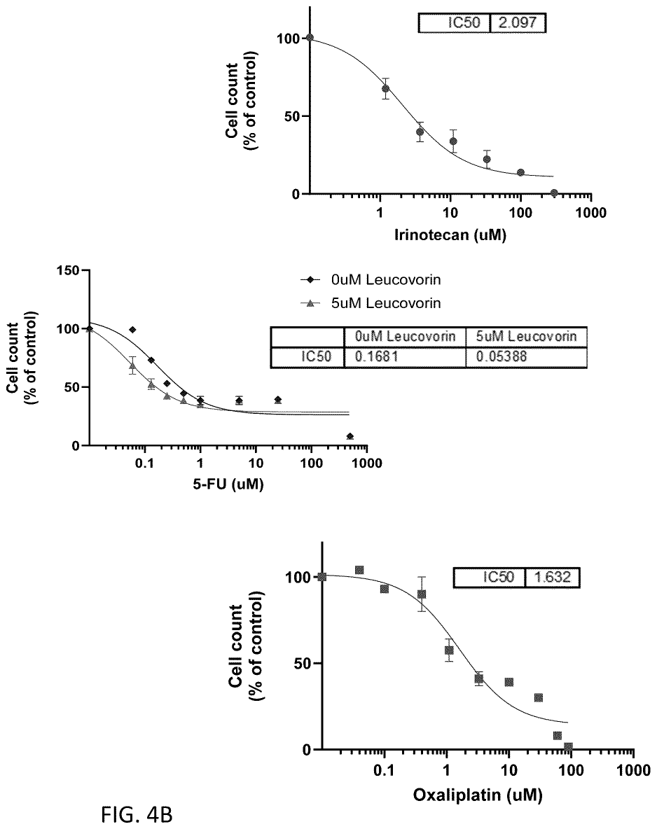

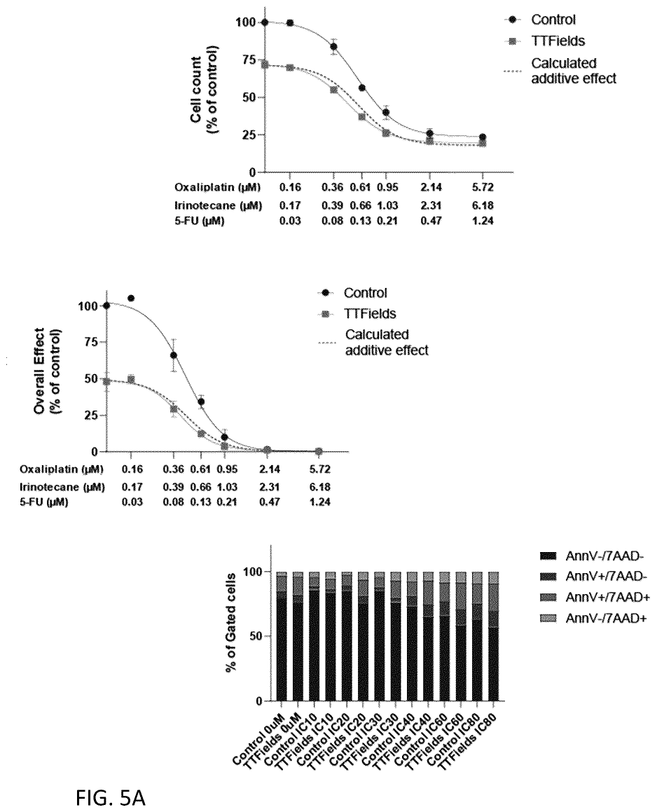

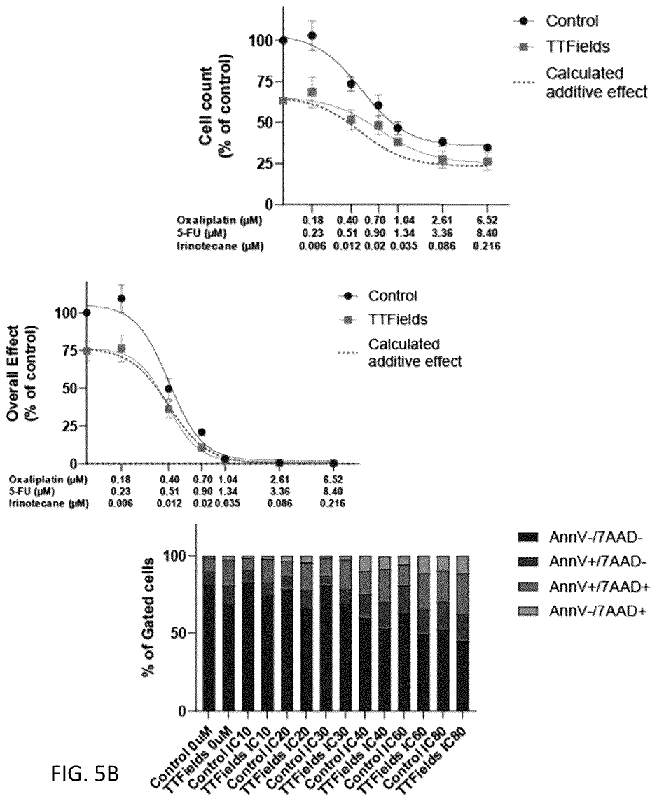

FIG. 3 shows the study design for the use of FOLFIRINOX and TTFields.

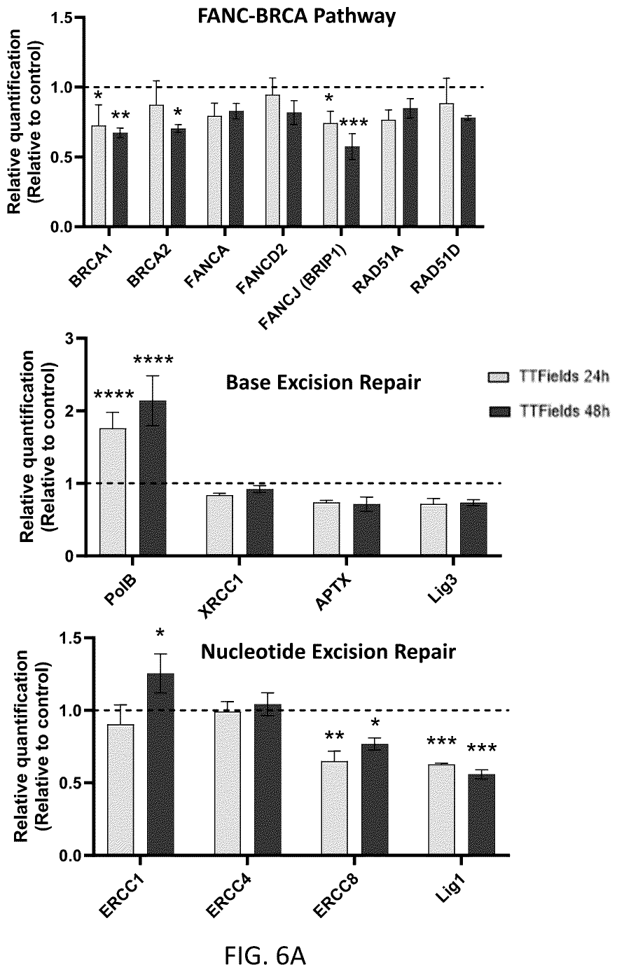

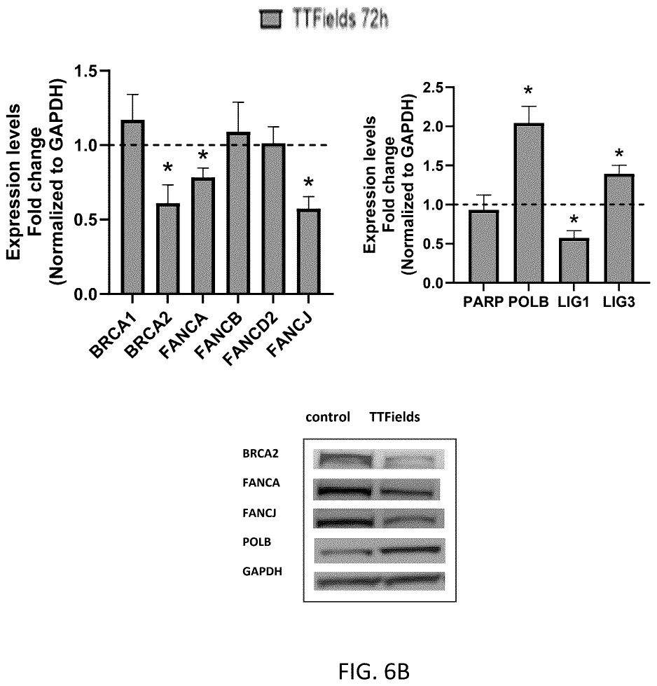

FIG. 6 shows TTFields modify the expression of DNA damage repair pathways. The FANC-BRCA pathway, Base Excision Repair pathway, and Nucleotide Excision Repair, Pathway were all examined for gene expression after exposure to 24 hr or 48 hr of TTFields. There was a decrease in expression of a few FANC-BRCA pathway genes and Nucleotide Excision Repair pathway genes. FIG. 6 also shows an increase in expression of PolB from the Base Excision Repair pathway genes. Protein levels were also examined and a decrease in protein levels of BRCA2, FANCA, and FANCJ were seen while an increase in protein levels of PolB were seen. Thus, the gene expression data was similar to the protein level data.

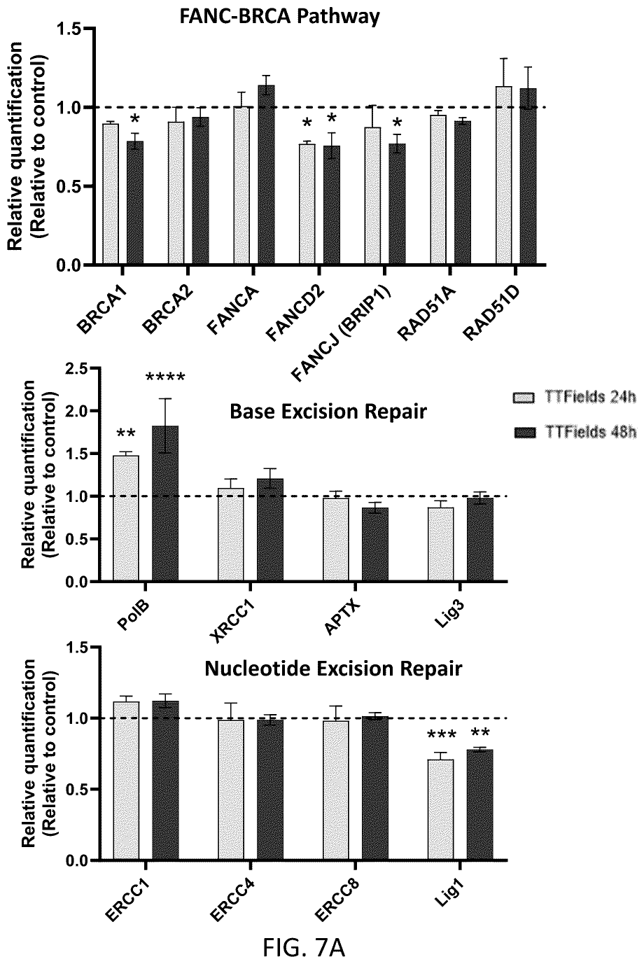

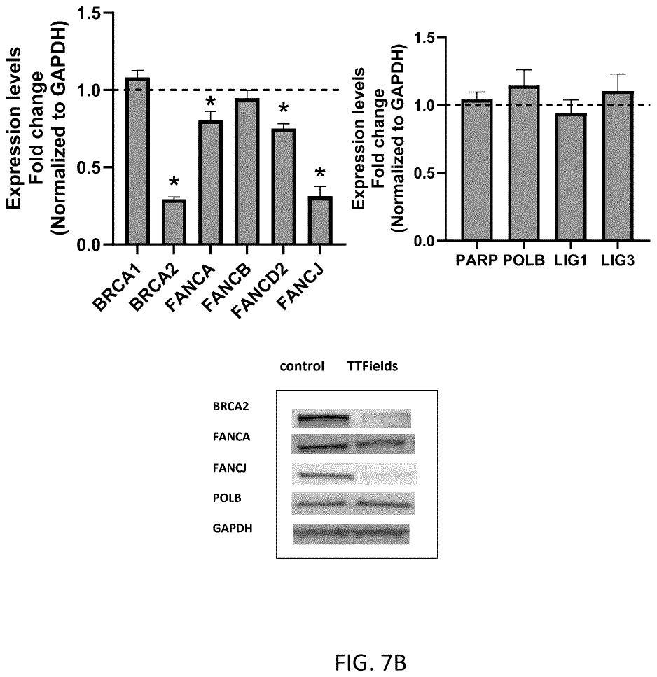

FIG. 7 is the same experiment as FIG. 6 except in a different cell line. Similar results were achieved in both the AsPC1 and BxPC3 cell lines.

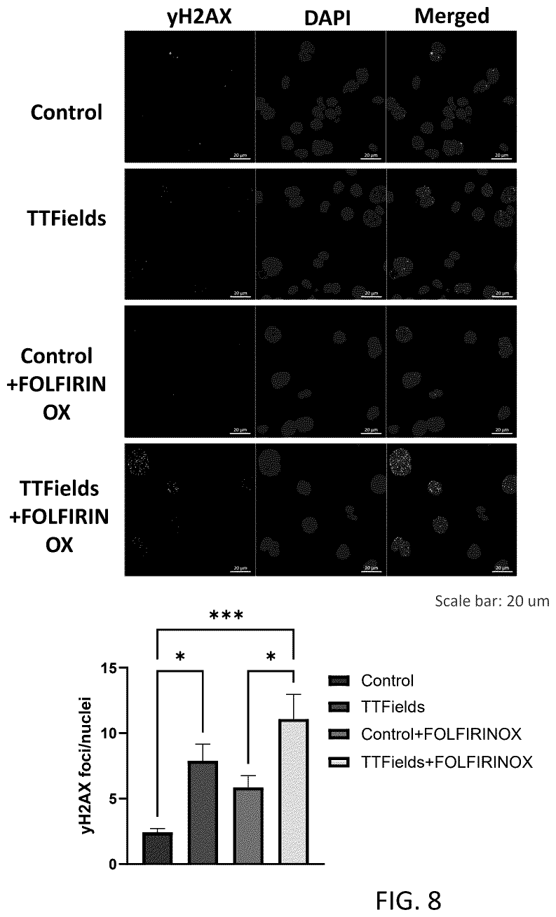

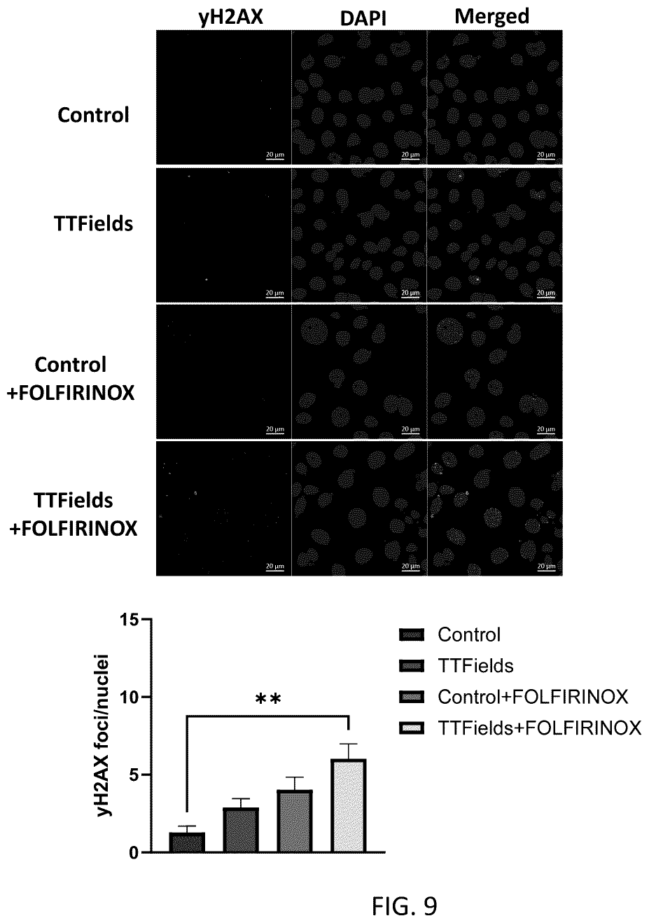

FIGS. 8 and 9 show TTFields increase DNA damage induced by FOLFIRINOX in two different pancreatic cancer cell lines.

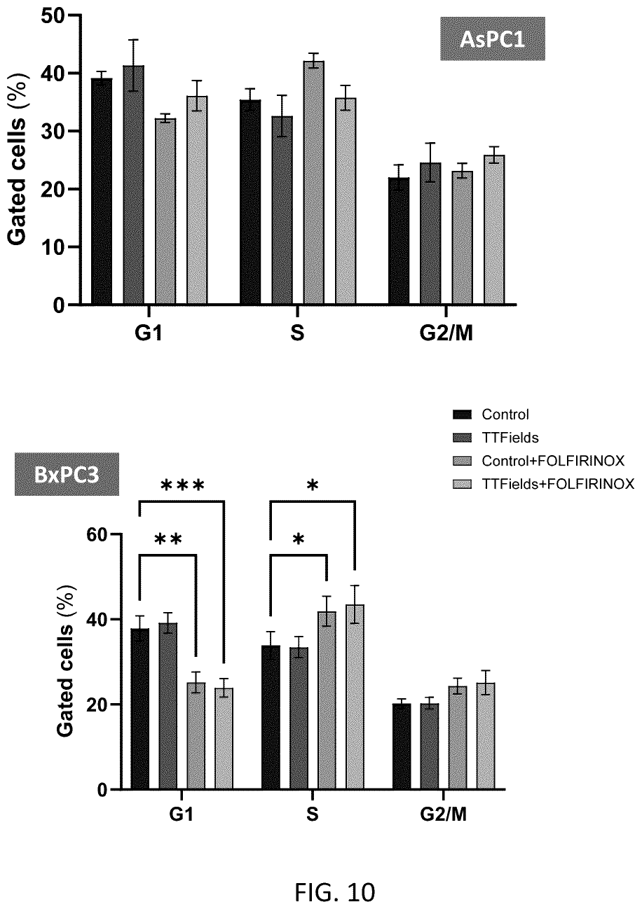

FIG. 10 shows FOLFIRINOX induce S-phase arrest, while TTFields have mild effect on cell cycle.

There appears to be a decrease in proliferation in the AsPC cell line and not the BxPC3 cell line. Since both cell lines are BRCA wild type and show downregulation of BRCA it is likely that BRCA expression does not have an effect on cytotoxicity concomitant to TTFields

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject in need thereof comprising:
a. applying an alternating electric field, at a frequency for a period of time, to a target site of the subject in need thereof; and
b. administering a combination of Leucovorin calcium, Fluorouracil, Irinotecane hydrochloride, Oxaliplatin to the subject in need thereof.

2. The method of claim 1, wherein the subject has cancer.

3. The method of claim 3, wherein the cancer is pancreatic cancer, lung cancer, breast cancer, cervical cancer, colon cancer, gastric cancer, renal cancer, prostate cancer, thyroid cancer, glioblastoma, mesothelioma, non-small cell lung, ovarian cancer, hepatic cancer.

4. The method of claim 1, wherein the target site comprises one or more cancer cells.

5. The method of claim 3, wherein the cancer has metastasized.

6. The method of claim 1, wherein one or more cancer cells are killed.

7. A method of reducing viability of cancer cells comprising:
a. applying an alternating electric field, at a frequency for a period of time, to a population of cancer cells in need thereof; and
b. contacting the population of cells with a combination of Leucovorin calcium, Fluorouracil, Irinotecane hydrochloride, Oxaliplatin.

8. A method of increasing apoptosis of cancer cells comprising:
a. applying an alternating electric field, at a frequency for a period of time, to a population of cancer cells; and
b. contacting the population of cancer cells with a combination of Leucovorin calcium, Fluorouracil, Irinotecane hydrochloride, Oxaliplatin.

9. The method of claim 5, wherein the cancer cells lack BRCA mutations.

10. The method of claim 1, wherein the alternating electric field reduces expression of the BRCA DNA damage repair pathway.

11. The method of claim 1, wherein the alternating electric field is applied before, after, or simultaneously with administering the combination of Leucovorin calcium, Fluorouracil, Irinotecane hydrochloride, Oxaliplatin.

12. The method of claim 1, wherein at least a portion of the applying an alternating electric field is performed after the administering a combination of Leucovorin calcium, Fluorouracil, Irinotecane hydrochloride, and Oxaliplatin, is performed before the administering a combination of Leucovorin calcium, Fluorouracil, Irinotecane hydrochloride, and Oxaliplatin, or is performed simultaneously with at least a portion of the administering the combination of Leucovorin calcium, Fluorouracil, Irinotecane hydrochloride, and Oxaliplatin.

13. The method of claim 1, wherein at least a portion of the applying an alternating electric field is performed before the Leucovorin calcium, Fluorouracil, Irinotecane hydrochloride, and Oxaliplatin are eliminated from the subject's body.

14. The method of claim 1, wherein the applying an alternating electric field has a duration of at least 12, 24, 36, 48, 60 or 72 hours.

15. The method of claim 1, wherein step b) is performed 1, 2, 3, 4, 5, 6, 7, 8, 9, or 10 days after step a) is performed.

16. The method of claim 1, wherein the frequency of the alternating electric field is between 50 kHz and 1 MHz.

17. The method of claim 1, wherein the alternating electric field has a field strength of 0.5 to 10 V/cm in at least a portion of the target region.

18. The method of claim 1, wherein the combination of Leucovorin calcium, Fluorouracil, Irinotecane hydrochloride, and Oxaliplatin is administered in a dosing regimen comprising administering oxaliplatin at a dose of 85 mg per square meter, given as a 2-hour intravenous infusion, immediately followed by leucovorin at a dose of 400 mg per square meter, given as a 2-hour intravenous infusion, and, after 30 minutes, administering irinotecan at a dose of 180 mg per square meter, given as a 90-minute intravenous infusion, and immediately followed by administering fluorouracil at a dose of 400 mg per square meter, by intravenous bolus, followed by a continuous intravenous infusion of 2400 mg per square meter of fluorouracil over a 46-hour period every 2 weeks.

19. The method of claim 1, wherein cell volume is increased, clonogenicity is reduced, tumor volume is decreased, and/or the frequency of abnormal mitotic events in the cells are increased.

20. The method of claim 1, wherein an inflammatory response is induced in the subject.
